SEQUENCE LISTING (12 sequences)

SEQ ID NO: 1 - protein, 5 aa, Artificial Sequence
  Anthrax Inhibitor Peptides
QLDHS

SEQ ID NO: 2 - protein, 5 aa, Artificial Sequence
  Anthrax Inhibitor Peptides
STDHS

SEQ ID NO: 3 - protein, 12 aa, Artificial Sequence
  Anthrax Inhibitor Peptides
AWPLSQLDHSYN

SEQ ID NO: 4 - protein, 12 aa, Artificial Sequence
  Anthrax Inhibitor Peptides
YHLSSQQLDHSL

SEQ ID NO: 5 - protein, 13 aa, Artificial Sequence
  Anthrax Inhibitor Peptides
SPHGSTDHSTTAY

SEQ ID NO: 6 - protein, 12 aa, Artificial Sequence
  Anthrax Inhibitor Peptides
SPHGSTDHSTTA

SEQ ID NO: 7 - protein, 7 aa, Artificial Sequence
  Anthrax Inhibitor Peptides
STDSGWV

SEQ ID NO: 8 - protein, 9 aa, Artificial Sequence
  Anthrax Inhibitor Peptides
CTSTDATYC

SEQ ID NO: 9 - protein, 9 aa, Artificial Sequence
  Anthrax Inhibitor Peptides
CPSSTLFAC

SEQ ID NO: 10 - protein, 12 aa, Artificial Sequence
  Anthrax Inhibitor Peptides
ATWGHPRSSQGM

SEQ ID NO: 11 - protein, 7 aa, Artificial Sequence
  Anthrax Inhibitor Peptides
STDHSLY

SEQ ID NO: 12 - protein, 12 aa, Artificial Sequence
  PA63 Binding Peptide
HTSTYWWLDGAP

(19) United States
(12) Patent Application Publication
(10) Pub. No.: US 2009/0054317 A1
(43) Pub. Date: Feb. 26, 2009

(54) POLYVALENT INHIBITORS OF PATHOGENS

(76) Inventors: Ravindra S. Kane, Niskayuna, NY (US); Jeremy Mogridge, Toronto (CA); Prakash R. Rai, Troy, NY (US); Saleem Basha Shaik, Cincinnati, OH (US)

Correspondence Address: ELMORE PATENT LAW GROUP, PC, 515 Groton Road, Unit 1R, Westford, MA, 01886, US

(21) Appl. No.: 11/838,408

(22) Filed: Aug. 14, 2007

Related U.S. Application Data
(60) Provisional application No. 60/837,503, filed on Aug. 14, 2006.

(51) Int. Cl. A61K 38/00; C07K 7/00; C40B 30/04; A61P 31/00

(52) U.S. Cl. 514/12; 530/330; 530/328; 530/327; 530/326; 530/325; 530/324; 530/329; 514/2; 514/13; 514/14; 514/15; 514/16; 514/17; 514/18; 506/9

(57) ABSTRACT

The present invention provides polyvalent host cell receptor-directed pathogen inhibitors and methods for designing and using such polyvalent inhibitors. Polyvalent inhibitors of the invention comprise a biocompatible scaffold having attached thereto a plurality of ligands, e.g. peptides, capable of binding a host cell receptor or portion thereof of a pathogen or toxin of a pathogen thereby preventing the pathogen or toxin thereof from binding to the host cell and causing toxicity of the host cell. Also provided are methods of designing such polyvalent host cell receptor-directed pathogen inhibitors and methods of using such inhibitors to prevent and/or treat infections of a host. The compositions of the inventions can be useful for treating anthrax.

### RELATED APPLICATION

This application claims the benefit of U.S. Provisional Application No. 60/837,503, filed on Aug. 14, 2006. The entire teachings of the above application are incorporated herein by reference.

### GOVERNMENT SUPPORT

The invention was supported, in whole, or in part, by NIH grant number R21 AI053506. The Government has certain rights in the invention.

### BACKGROUND OF THE INVENTION

Pathogens can develop resistance to drugs directed against microbial targets by modifying the drug, by lowering the concentration of drug that reaches the target, or by mutating the target (Levy, S. B. & Marshall, B. (2004) Nat. Med. 10, S122-S129; Hogan, D. & Kolter, R. (2002) Curr. Opin. Microbiol. 5, 472-477). There is also an increasing concern that therapeutics developed for bioterrorism agents may be rendered ineffective if the microbial target is altered intentionally. This problem could be overcome, however, by designing antimicrobials that block host proteins used by the pathogen or its toxins to cause disease.

Microbial pathogens and their products interact with host structures to facilitate colonization or to promote cellular uptake. Many of these interactions are polyvalent, meaning that they involve the simultaneous binding of multiple ligands on one entity to multiple receptors on another (Mammen, M., et al. (1998) Angew. Chem. Int. Ed. 37, 2754-2794). The design of synthetic polyvalent (Mammen, M., et al. (1995) J. Med. Chem. 38, 4179-4190; Mochalova, L. V., et al. (1994) Antiviral Res. 23, 179-190; Yarema, K. J. & Bertozzi, C. R. (1998) Curr. Opin. Chem. Biol. 2, 49-61; Kamitakahara, H., et al. (1998) Angew. Chem. Int. Ed. 37, 1524-1528; Mourez, M., Kane, et al. (2001) Nat. Biotechnol. 19, 958-961) or oligovalent (Kitov, P. I., et al. (2000) Nature 403, 669-672; Fan, E., et al. (2000) J. Am. Chem. Soc. 122, 2663-2664) molecules also represents a promising approach to enhance the potency of inhibitors of microbial pathogens and toxins. Current examples of this approach have involved the design of molecules that bind directly to the pathogen or toxin. Inhibitors that bind host proteins would represent an effective way to attenuate virulence that may be less susceptible to resistance mechanisms; and the use of polyvalency could provide a significant enhancement in the potency of these inhibitors.

ANTXR1 and ANTXR2 are host receptors that bind and internalize anthrax toxin (Bradley, K. A., et al. (2001) Nature 414, 225-229; Scobie, H. M., et al. (2003) Proc. Natl. Acad. Sci. USA 100, 5170-5174). These proteins are likely important for anthrax pathogenesis because the toxin impairs the immune response and is responsible for the major symptoms and death associated with anthrax. Thus, blocking these receptors could represent a promising approach to anthrax therapy.

ANTXR1 and ANTXR2 are widely-expressed type I membrane proteins that bind components of the extracellular matrix (Bell, S. E., et al. (2001) J. Cell Sci. 114, 2755-2773). They both contain an extracellular I domain, which binds the protective antigen (PA) component of anthrax toxin. The two proteins are 40% identical overall and share 60% identity within their I domains. The PA-receptor interaction is mediated through an aspartate side chain of PA that completes the coordination of a metal ion bound by a metal ion dependent adhesion site (MIDAS) of the receptor I domain (Santelli, E., et al. (2004) Nature 430, 905-908; Lacy, D. B., et al. (2004) Proc. Natl. Acad. Sci. USA 101, 6367-6372). PA is processed by a protease into a 63 kDa fragment, PA₆₃, which oligomerizes into ring-shaped heptamers (Collier, R. J. & Young, J. A. T. (2003) Annu. Rev. Cell Dev. Biol. 19, 45-70). Heptameric PA₆₃ ([PA₆₃]₇) binds the enzymatic components of the toxin, edema factor (EF) and lethal factor (LF), and the resulting complexes are internalized. Upon reaching an acidic compartment, [PA₆₃]₇ dissociates from its receptors and inserts flexible loops into the membrane to form a beta-barrel pore (Rainey, G. J. A., et al. (2005) Proc. Natl. Acad. Sci. USA 102, 13278-13283). Insertion of [PA₆₃]₇ that has been internalized by ANTXR1 occurs at a higher pH found in early endosomes compared to ANTXR2-internalized [PA₆₃]₇, which inserts only at a lower pH found in late endosomes—this difference likely results from the higher affinity of the PA-ANTXR2 interaction. The pore that forms as a result of [PA₆₃]₇ insertion allows EF and LF to translocate across the endosomal membrane (Krantz, B. A., et al. (2005) Science 309, 777-781). EF is an adenylate cyclase and LF is a protease that cleaves mitogen activated protein kinase kinases. The enzymatic activities of these proteins contribute to disease progression in several ways and at different stages of infection (Collier, R. J. & Young, J. A. T. (2003) Annu. Rev. Cell Dev. Biol. 19, 45-70).

Several anthrax toxin inhibitors have been described that interfere with different steps in this intoxication pathway (Mourez, M., Kane, et al. (2001) Nat. Biotechnol. 19, 958-961; Bradley, K. A., et al. (2001) Nature 414, 225-229; Scobie, H. M., et al. (2003) Proc. Natl. Acad. Sci. USA 100, 5170-5174; Collier, R. J. & Young, J. A. T. (2003) Annu. Rev. Cell Dev. Biol. 19, 45-70; Rainey, G. J. A. & Young, J. A. T. (2004) Nat. Rev. Microbiol. 2, 721-726; Karginov, V. A., et al. (2005) Proc. Natl. Acad. Sci. USA 102, 15075-15080; Sellman, B. R., et al. (2001) Science 292, 695-697; Maynard, J. A., et al. (2002) Nat. Biotechnol. 20, 597-601; Numa, M. M. D., et al. (2005) ChemBioChem 6, 1002-1006; Panchal, R., et al. (2004) Nat. Struc. Mol. Bio. 11, 67-72; Rai, P., et al. (2006) Nat. Biotechnol. 24, 582-586), but none has targeted the host receptors.

### SUMMARY OF THE INVENTION

The present invention provides polyvalent host cell receptor-directed pathogen inhibitors and methods for designing and using such polyvalent inhibitors. Polyvalent inhibitors of the invention comprise a biocompatible scaffold having attached thereto a plurality of ligands, e.g. peptides, carbohydrates, and small molecules, capable of binding a host cell receptor or portion thereof of a pathogen or toxin of a pathogen thereby preventing the pathogen or toxin thereof from causing toxicity to the host cell. Also provided are methods of designing such polyvalent host cell receptor-directed pathogen inhibitors and methods of using such inhibitors to prevent and/or treat infections of a host. The compositions of the inventions can be useful for treating against bacillus, Escherichia, staphylococcus, pneumococcus, and other bacteria, parasites, fungi, yeast, viral and protozoan infections. In a preferred embodiment, the compositions of the invention can be used to treat anthrax.

### BRIEF DESCRIPTION OF THE DRAWINGS

FIG. 1: Characterization of AWPLSQLDHSYN-functionalized polyvalent inhibitors in vitro. (A) Inhibition of anthrax toxin-induced RAW264.7 cytotoxicity by polyvalent liposomes presenting AWPLSQLDHSYN (▪) or control thioglycerol-functionalized liposomes (). (B) Phage presenting the sequence AWPLSQLDHSYN, which was isolated by panning against ANTXR1, binds specifically to both ANTXR1 and ANTXR2, but does not bind to BSA. (C) Binding of fluorescein-incorporating liposomes presenting the sequence AWPLSQLDHSYN (black bars), the sequence HTSTYWWLDGAP (white bars), and thioglycerol (gray bars) to ANTXR2, [PA₆₃]₇, PA, and BSA. (D) Binding of fluorescein-containing liposomes presenting AWPLSQLDHSYN (black bars) and thioglycerol-functionalized control liposomes (gray bars) to CHO-R1-ANTXR1, CHO-R1-ANTXR2, and receptor-deficient CHO-R1.1 cells.

### DETAILED DESCRIPTION OF THE INVENTION

The present invention provides compositions and methods for treating an infection in a mammal by administering to the mammal a therapeutically effective amount of a polyvalent host cell receptor-directed inhibitor of the pathogen or toxin of the pathogen. The polyvalent inhibitor is comprised of multiple ligands, including peptides, carbohydrates, small molecules and the like, linked to a biocompatible scaffold. In one preferred embodiment, the scaffold of the polyvalent molecule is comprised of a liposome. In another preferred embodiment, the biocompatible scaffold comprises a polymer. The biocompatible scaffold also may be further functionalized to provide desired physical characteristics.

As used herein, a “polyvalent host cell-directed inhibitor of a pathogen or toxin of a pathogen” refers to a biocompatible scaffold that has at least one, and preferably a plurality of covalently linked ligands, preferably peptides, capable of binding a host cell receptor or portion thereof of a pathogen or toxin and preventing toxicity of the host cell. As used herein a plurality of ligands refers to at least 5 ligands, more preferably at least about ten ligands and most preferably at least about 25 ligand units. A polyvalent inhibitor of the invention may present a plurality of the same ligands or may present a plurality of ligands that differ. For example, in the case where the ligand is a peptide, one or more of the peptides can differ in the amino acid sequence. As used herein “multiple copies” of a peptide are at least 2, preferably at least 5 peptides comprising the same amino acid sequence.

In a preferred embodiment, the peptides comprising the polyvalent inhibitor are between about 3 and 30 amino acids in length, even more preferably about 5 and 15 amino acids in length and most preferably between about 7 and 13 amino acids in length. The ligands or peptides can optionally be modified by adding a cysteine or similar residue or reactive group to facilitate attachment to the scaffold molecule. Preferably, the reactive residue or group is attached to the N- or C-terminal of the peptide and is selected to minimize steric or other interference between the peptide (or ligand in the case of other ligand molecules) and the receptor.

The compositions and methods of the invention are particularly useful to treat against bacterial or other infections, including the treatment of or prevention against (collectively “treating”) infections with gram-negative bacteria or gram-positive bacteria. The compositions of the invention can be particularly effective for treating aspergillus, bacillus, blastomycosis, bordetello, burkholdia, campylobacter, candida, Chlamydia, clostridium, coccidioides, corynebacterium, cryptococcus, enterobacter, enterococcus, escherichia, haemophilus, helicobacter, klebsiella, legionella, mycobacterium, pseudomonas, pneumococcus, salmonella, staphylococcus, xanthomonas and vibrio and other bacteria, parasites, fungi, yeast, viral and protozoan infections. In a preferred embodiment, the compositions of the invention can be used to treat anthrax. The polyvalent inhibitor of the invention is particularly useful in treatment of patients infected with for example, gram positive or gram negative bacteria or bacteria that may be resistant to antibiotics. The present composition can also be administered to a mammal in need of such therapy in conjunction with other therapies such as antibiotics, chemotherapy and the like.

According to one embodiment of the invention, the polyvalent molecule inhibits attachment to host cell receptors and ligands by blocking the receptors and ligands of the host cell. Viral infections that may be treated by the compositions and methods of the invention include, but are not limited to: Herpes viruses, Hepatitisviruses, Retroviruses, Orthomyxoviruses, Paramyxoviruses, Togaviruses, Picornaviruses, Papovaviruses and Gastroenteritisviruses. Known viral receptors include but are not limited to: CD46 and signaling lymphocyte activation molecule (SLAM) used by measles virus; angiotensin-converting enzyme 2 receptor for the SARS virus; CCR5 receptor for human immunodeficiency virus (HIV); CXCR4 receptor for HIV; CD4 receptor for HIV.

According to another embodiment of the invention, the polyvalent molecule is specific for the host cell receptors of human or domestic animal bacterial pathogens or toxins of such pathogens. Bacteria causing serious human diseases treatable by the compositions and methods of the invention include but are not limited to: Bacillus anthracis, Corynebacterium diptheriae, Bordetella pertussis, Vitrio cholerae, Clostridium botulinum, Clostridium tetani, enterohemorrhagic Escherichia coli, Staphylococcus aureus, Staphylococcus epidermidis, Enterococcus faecalis and E. faecium, Streptococcus pneumoniae, Pseudomonas aeruginosa, Burkholdia cepacia, Xanthomonas maltophila, Escherichia coli, Enterobacter spp, Klebsiella pneumoniae and Salmonella spp. Known receptors for bacterial pathogens or toxins of such pathogens include, but are not limited to, ANTXR1 and ANTXR2 receptors for anthrax toxin and CD44 receptor for Group A Streptococcus. The invention is particularly useful for treating drug resistant bacterial infection or infection by other drug resistant pathogens as the polyvalent inhibitor of the invention targets the host cell receptors thereby bypassing the mutation mechanism of the pathogen to develop drug resistance to the inhibitors of the invention.

According to one embodiment of the invention, the polyvalent molecule is specific for host cell receptors associated with protozoa infecting humans and causing human diseases. Protozoan organisms causing human diseases according to the present invention include but are not limited to Malaria e.g. Plasmodium falciparum and M. ovale, Trypanosomiasis (sleeping sickness) e.g. Trypanosoma cruzei, Leischmaniasis e.g. Leischmania donovani, Amebiasis e.g. Entamoeba histolytica.

According to one embodiment of the invention, the polyvalent molecule is specific for the host cell receptors associated with fungi causing pathogenic infections in humans. Particularly preferred fungi causing human diseases according to the present invention include (but not restricted to) Candida albicans, Histoplasma neoformans, Coccidioides immitis and Penicillium marneffei.

The ligands attached to the biocompatible backbone have the ability to interfere with binding of a pathogen or its toxin to at least one receptor specific for the pathogen or toxin. Preferred compositions of the invention comprise a plurality of peptides of the same sequence linked to the scaffold. However, multiple peptides of differing sequence also may be suitably linked to a scaffold. Such an approach may be preferred e.g. where more than one type of infectious disease agent produces multiple toxins. Preferably, more than one type of polyvalent toxin inhibitor can be administered during the course of treatment.

Peptides that have the ability to bind the host cell's receptor or a portion of the receptor specific for a pathogen or toxin can be identified by use of phage display techniques. These techniques are well known in the art. See, for example, Gordon et al., Nature, 395:710-713, 1998; Smith et al, Science 228:1315 (1985). Phage screening kits are also available commercially. For example, a commercial library of peptides displayed on the surface of a bacteriophage M13 can be purchased from New England Biolabs. Screening peptide libraries is a proven strategy for identifying inhibitors of protein-ligand interactions.

Upon identification of a host cell receptor of a pathogen or a toxin, that receptor or any portion thereof may be used to screen for peptides that bind to the host cell receptor or portion thereof using phage display. In general, phage display libraries using, e.g., M13 or fd phage, are prepared using conventional procedures such as those described in the foregoing reference. The libraries display inserts containing from 4 to 80 amino acid residues. The inserts optionally represent a completely degenerate or a biased array of peptides. Ligands that bind selectively to a known host cell receptor site for a pathogen or toxin are obtained by selecting those phages which express on their surface an amino acid sequence which recognizes and binds to the receptor. These phages then are subjected to several cycles of reselection to identify the receptor binding phages that have the most useful binding characteristics. The minimal linear portion of the sequence that binds to the receptor ligand binding site can be determined. Typically, phages that exhibit the best binding characteristics (e.g., highest affinity) are further characterized by nucleic acid analysis to identify the particular amino acid sequences of the peptides expressed on the phage surface and the optimum length of the expressed peptide to achieve optimum binding to the receptor binding site of the ligand or the ligand binding site of the receptor located on the host cell.

Peptides and other ligands can be screened for their ability to bind to a host cell pathogen or toxin receptor in vitro using standard binding assays well known to the ordinary artisan and described below. A host cell receptor for a pathogen or toxin of a pathogen may be presented in a number of ways including but not limited to cells expressing the receptor (such as those described below), an isolated extracellular domain of the receptor (e.g. the I domain of host cell receptors for anthrax toxin, ANTXR1 and/or ANTXR2), a fragment thereof or a fusion protein of the extracellular domain of the host cell toxin or pathogen receptor or portion thereof and another protein such as an immunoglobulin. For some high throughput screening assays the use of purified forms of the receptor, its extracellular domain or a fusion of its extracellular domain with another protein may be preferable. Isolation of binding partners may be performed in solution or in solid state according to well-known methods.

Standard binding assays are well known in the art, and a number of these are suitable in the present invention including ELISA, competition binding assay, sandwich assays, radioreceptor assays using radioactive labels (e.g., where the binding is blocked in the presence of the ligand), labeled in vitro protein-protein binding assays, electrophoretic mobility shift assays, immunoassays, cell-based assays such as two- or three-hybrid screens, etc. The nature of the assay is not essential provided it is sufficiently sensitive to detect binding of a small number of library members.

One preferred biocompatible scaffold in accordance with the invention are liposomes. The liposome composition of the present invention preferably comprises vesicle-forming lipids. Examples of vesicle-forming lipids (a) form spontaneously into bilayer vesicles in water, as exemplified by phospholipids, and/or (b) stably incorporate into lipid bilayers, with its hydrophobic moiety in contact with the interior, hydrophobic region of the bilayer membrane, and its head group moiety oriented toward the exterior, polar surface of the membrane.

The vesicle-forming lipids of this type are preferably ones having two hydrocarbon chains, typically acyl chains, and a head group, either polar or nonpolar. There are a variety of synthetic vesicle-forming lipids and naturally-occurring vesicle-forming lipids, including the phospholipids, such as phosphatidylcholine, phosphatidylethanolamine, phosphatidic acid, phosphatidylinositol, and sphingomyelin, where the two hydrocarbon chains are typically between about 14-22 carbon atoms in length, and have varying degrees of unsaturation. The above-described lipids and phospholipids whose acyl chains have varying degrees of saturation can be obtained commercially or prepared according to published methods. Other suitable lipids include glycolipids and sterols such as cholesterol.

Preferred diacyl-chain lipids for use in the present invention include diacyl glycerol, phosphatidyl ethanolamine (PE) and phosphatidyl glycerol (PG). These lipids are preferred for use as the vesicle-forming lipid, the major liposome component, and for use in the derivitized lipid described below.

The scaffold can also preferably comprise pegylated lipids, such as those reviewed in Rovira-Bru et al., Biophysical Journal, Vol. 83(5) 2419-2439 (2002), which is incorporated herein by reference. The references cited therein are also incorporated herein by reference. Thus, in a preferred embodiment, the scaffold is a liposome comprising a mixture of lipids and pegylated lipids.

Additionally, the vesicle-forming lipid is selected to achieve a specified degree of fluidity or rigidity, to control the stability of the liposome in serum. The rigidity of the liposome, as determined by the vesicle-forming lipid, may also play a role in presentation of the liposome to a target host cell exhibiting the receptor of the pathogen or toxin of the pathogen.

Liposomes having a more rigid lipid bilayer, or a liquid crystalline bilayer, are achieved by incorporation of a relatively rigid lipid, e.g., a lipid having a relatively high phase transition temperature, e.g., up to 60° C. Rigid, or saturated, lipids contribute to greater membrane rigidity in the lipid bilayer. Other lipid components, such as cholesterol, are also known to contribute to membrane rigidity in lipid bilayer structures. On the other hand, lipid fluidity is achieved by incorporation of a relatively fluid lipid, typically one having a lipid phase with a relatively low liquid to liquid-crystalline phase transition temperature, e.g., at or below room temperature.

The lipids forming the bilayer vesicle or liposome, can also be cationic lipids, which have a lipophilic moiety, such as a sterol, an acyl or diacyl chain, and where the lipid has an overall net positive charge. Preferably, the head group of the lipid carries the positive charge. Exemplary cationic lipids include 1,2-dioleyloxy-3-(trimethylamino) propane (DOTAP); N-[1-(2,3,-ditetradecyloxy)propyl]-N,N-dimethyl-N-hydroxyethylammonium bromide (DMRIE); N-[1-(2,3,-dioleyloxy)propyl]-N,N-dimethyl-N-hydroxy ethylammonium bromide (DORIE); N-[1-(2,3-dioleyloxy) propyl]-N,N,N-trimethylammonium chloride (DOTMA); 3-β-[N—(N′,N′-dimethylaminoethane)carbamoly]cholesterol (DC-Chol); and dimethyldioctadecylammonium (DDAB).

The cationic vesicle-forming lipid may also be a neutral lipid, such as dioleoylphosphatidyl ethanolamine (DOPE) or an amphipathic lipid, such as a phospholipid, derivitized with a cationic lipid, such as polylysine or other polyamine lipids. For example, the neutral lipid (DOPE) can be derivitized with polylysine to form a cationic lipid.

Preferably, a plurality of lipid head groups in the liposome are functionalized with a reactive group that is capable of binding to or reacting with the ligand. For example, where the ligand is characterized by a terminal cysteine residue, the liposome can be characterized by at least one lipid having a group capable of reacting with the cysteine thiol. Examples of such reactive groups include thiols, disulfides and chloroacetyl groups. Where the liposome comprises a pegylated lipid, the ligand can be bound to the terminus of the polyethyleneglycol group, such as through a disulfide linkage. In a preferred embodiment, the ligands are not encapsulated within the liposome, but are external to it.

In one preferred embodiment, the biocompatible scaffold is a polymer. Polymer backbones which are suitable for the present invention include backbones with low intrinsic toxicity. Suitable polymer backbones include, for example, water soluble polymers such as polyhydroxypropylmethacrylamide, polyethylene glycol, copolymers of ethylene glycol/propylene glycol, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propylene glycol homopolymers, polypropylene oxide/ethylene oxide co-polymers, polyoxyethylated polyols and polyvinyl alcohol; chitin/chitosan, cellulose; polypeptides comprising natural or synthetic amino acid residues such as, for example, polylysine, polyamides, polyglutamic acid, and polyaspartic acid; oligonucleotides such as, for example, DNA and RNA; polycarbohydrates or polysaccharides such as, for example, polyamylose, polyfuranosides, polypyranosides, carboxymethylamylose, and dextrans; polystyrenes such as, for example, chloromethylated polystyrene and bromomethylated polystyrene; polyacrylamides such as, for example, polyacrylamide hydrazide; polyacids such as, for example, polyacrylic acid; polyols such as, for example, polyvinyl alcohol; polyvinyls such as, for example, polyvinyl chloride and polyvinyl bromide; polyesters; polyurethanes; polyolefins; polyethers; and the like as well as other monomeric, polymeric or oligomeric materials containing reactive functional groups along the length of their chain which can be substituted with a phosphorothioate monoester group. Polyethylene glycol propionaldehyde may have advantages in manufacturing due to its stability in water.

The polymers of the present invention can be prepared via, direct polymerization or copolymerization of a monomer, and nucleophilic side chain substitution on an activated polymer. The monomers can be polymerized using, for example, methods of free radical polymerization which are well known in the art. Due to reactivity differences between the two monomers, the mole ratio of the monomers in the copolymer product can be different from the mole ratio of the monomers in the initial reaction mixture. This reactivity difference can also result in a non-random distribution of monomers along the polymer chain.

The polymer may be of any molecular weight, and may be branched or unbranched. For polyethylene glycol, the preferred molecular weight is between about 2 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

The polyvalent inhibitor of the invention can be administered by injection, e.g., intravenously, subcutaneously, or intramuscularly by a suitable mechanical device, such as hypodermic needle and syringe, air gun injection devices, or topically, vaginally, rectally, via inhalation devices, etc., at a dosage of about 1 mg/kg/day to about 10 g/kg/day of active agent depending upon the individual patient. As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin Remington's Pharm. Sci., 15^{th} Ed. (Mack Publ. Co., Easton (1975)). Pharmaceutically acceptable carriers are sterile and pyrogen-free.

The quantity of an inhibitor to be administered will be determined on an individual basis and will be determined, at least in part, by consideration of the individual's size, the severity of symptoms to be treated and the result sought.

The polyvalent inhibitor can be administered as a solid or in solution, for example, in aqueous or buffered aqueous solution. The inhibitor can be administered alone or in a pharmaceutical composition comprising the inhibitor with an acceptable carrier or diluent and, optionally, one or more additional drugs. Such additional drugs include but are not limited to antibiotics, antivirals and chemotherapy.

The polyvalent inhibitors of the invention are useful in methods to prevent or treat conditions in patients that result from infection by a pathogen and/or subsequent contact with the pathogen or any toxin of such pathogen. In accordance with this aspect of the invention, a therapeutically effective amount of a polyvalent inhibitor of the invention is administered to a patient in need of prevention or treatment of a condition caused by a pathogen or a toxin of a pathogen. As used herein, “patient” refers to any animal or mammal, and includes but is not limited to, domestic animals, sports animals, primates and humans; more particularly, the term refers to humans. As used herein, the term “effective amount” means the amount needed to achieve the desired therapeutic or diagnostic effect or efficacy. The actual effective amounts of drug can vary according to the specific drug or combination thereof being utilized, the particular composition formulated, the mode of administration, and the age, weight, condition of the patient, and severity of the symptoms or condition being treated. The terms “treating”, “treatment” and the like are used herein to mean affecting a subject, tissue or cell to obtain a desired pharmacologic and/or physiologic effect. The effect may be prophylactic in terms of completely or partially preventing a condition. Examples of such conditions and the pathogens or toxins responsible for such conditions which may be treated by the compositions and methods of the invention include but are not limited to those listed in Table A:

TABLE A

| Organism/toxin | Target | Disease |
|---|---|---|
| Damage membranes | | |
| Aeromonas | Glycophorin | Diarrhea |
| hydrophila/aerolysin | | |
| Clostridium | Cholesterol | Gas gangrene and others |
| perfringens/perfringolysin O | | |
| Escherichia coli/ | Plasma membrane | Urinary tract infections |
| hemolysin (also produced by | | |
| other genera of bacteria) | | |
| Listeria monocytogenes/ | Cholesterol | Foodborne systemic/illness |
| listeriolysin O | | meningitis |
| Staphyloccocus aureus/ | Plasma membrane | Abscesses and others |
| α-toxin | | |
| Streptococcus | Cholesterol | Pneumonia and others |
| pneumoniae/pneumolysin | | |
| Streptococcus | Cholesterol | Strep throat, scarlet fever and |
| pyogenes/streptolysin O | | others |
| Inhibit protein synthesis | | |
| Corynebacterium | Elongation factor 2 | Diphtheria |
| diphtheriae/diphtheria toxin | | |
| E. coli/Shigella | 28S rRNA | Hemmohagic colitis and |
| dysenteriae/shiga toxins | | hemolytic remic syndrome |
| Pseudomonas | Elongation factor 2 | Pneumonia and other diseases |
| aeruginosa/exotoxin A | | |
| Activate Second Messanger | | |
| pathways | | |
| Cytotoxic necrotizing factor | Rho G-proteins | UTIs |
| (CNF) | | |
| Heat labile toxin (LT) | G-proteins | Diarrhea |
| Heat stable toxin (ST) | guanylate cyclase | Diarrhea |
| | receptor | |
| Cytolethal distending toxin | Unknown | Diarrhea |
| Enteroaggregative E. Coli heat | Unknown | Diarrhea |
| stable toxin (EAST) | | |
| Bacillus anthracis/ | ATP | Anthrax |
| edema factor | | |
| Bordetella pertussis/ | Rho G-proteins | Rhinitis |
| dermonecrotic toxin | | |
| Bordetella pertussis/pertussis | G-protein(s) | Pertussis |
| toxin | | |
| Clostridium botulinum/ | Monomeric G-actin | Botulism |
| C2 toxin | | |
| C. botulinum/C3 toxin | Rho G-protein | Botulism |
| Clostridium difficile/toxins A | Rho G-protein(s) | Diarrhea/antibiotic associated |
| and B | | pseudomembranous colitis |
| Vibrio cholerae/cholera | G-protein(s) | Cholera |
| toxin | | |
| Activate Immune Response | | |
| S. aureus/enterotoxins | TCR and MHC II | Food poisoning and other |
| | | diseases |
| S. aureus/exfoliative toxins | TCR and MHC II | Scalded skin syndrome and |
| | | others |
| S. aureus/toxic-shock toxin | TCR and MHC II | Toxic shock syndrome and |
| | | others |
| S. pyogenes/pyrogenic | TCR and MHC II | Scarlet fever and toxic shock |
| exotoxins | | syndrome |
| Protease | | |
| B. anthracis/lethal factor | MAPKK1/MAPKK2 | Anthrax |
| C. botulinum/neurotoxins | VAMP/synaptobrevin | Botulism |
| A-G | SNAP-25 syntaxin | |
| Clostridium tetani/ | VAMP/synaptobrevin | Tetanus |
| tetanus toxin | | |

Abbreviations in Table A:
TCR = T cell receptor;
MHC II = major histocompatibility comples class II;
MAPKK = mitogen-activated protein kinase kinase;
VAMP = vesicle assicated membrane protein;
SNAP-25 = synaptosomal-associated protein;

The methods of the invention are particularly useful in the treatment of conditions in a patient caused by multi-drug resistant bacteria or other multi-drug resistant pathogens.

Another aspect of the invention is directed to methods for designing polyvalent inhibitors of the invention. In accordance with this aspect of the invention, the method for designing a polyvalent pathogen inhibitor comprises the steps of:

(a) providing a purified host cell receptor or portion thereof of a pathogen or toxin of the pathogen;

(b) contacting the receptor of step (a) with a phage library displaying random peptides and isolating the phages that bind to the receptor of step (a);

(c) identifying the peptide associated with the phages isolated in step (b); and

(d) attaching multiple copies of the peptide identified in step (c) to a scaffold;

wherein the polyvalent inhibitor is capable of inhibiting the binding of a pathogen or a toxin of a pathogen to the host cell's receptor for the pathogen or toxin of the pathogen.

In a preferred aspect of the invention, host cell receptor-directed polyvalent inhibitors of anthrax toxin are provided for the prevention and treatment of anthrax toxicity in a host. Preferred polyvalent inhibitors are based on multiple copies of peptides capable of binding all or a portion of one or more of the host cell's receptors for anthrax, ANTXR1 and/or ANTXR2, are linked to a biocompatible scaffold. In a preferred embodiment, the biocompatible scaffold is a liposome.

In one embodiment, the invention comprises a polyvalent inhibitor of anthrax toxin comprising a biocompatible scaffold having attached thereto a plurality of peptides capable of binding all or a portion of at least one or both of the host cell receptors ANTXR1, ANTXR2. In one embodiment, at least one peptide, and preferably multiple copies of the peptide, bind to the I domain of ANTXR1 and/or ANTXR2. In one embodiment the peptide comprises the amino acid sequence DHS. In one embodiment, the peptide comprises the amino acid sequence STD. In one embodiment the peptide comprises the amino acid sequence QLDHS (SEQ ID NO: 1). In one embodiment the peptide comprises the amino acid sequence STDHS (SEQ ID NO: 2).

Preferred peptides capable of binding all or a portion of at least one or both of the host cell receptors ANTXR1, ANTXR2 are selected from the following peptides:

| | |
|---|---|
| AWPLSQLDHSYN, | (SEQ ID NO: 3) |
| YHLSSQQLDHSL, | (SEQ ID NO: 4) |
| SPHGSTDHSTTAY, | (SEQ ID NO: 5) |
| SPHGSTDHSTTA, | (SEQ ID NO: 6) |
| STDSGWV, | (SEQ ID NO: 7) |
| CTSTDATYC, | (SEQ ID NO: 8) |
| CPSSTLFAC, | (SEQ ID NO: 9) |
| ATWGHPRSSQGM, | (SEQ ID NO: 10) |
| and | |
| STDHSLY. | (SEQ ID NO: 11) |

In a preferred embodiment, the polyvalent inhibitor of the invention comprises multiple copies of a peptide having the amino acid sequence, AWPLSQLDHSYN (SEQ ID NO: 3). A detailed description of identification of the peptides suitable for use as a polyvalent inhibitor of anthrax toxin as well as the design of such polyvalent inhibitors is found in the examples which follow.

The following non-limiting examples are illustrative of the invention.

### EXAMPLE

### Results and Discussion

To develop an anthrax toxin inhibitor, we first used phage display (Mourez, M., Kane, et al. (2001) Nat. Biotechnol. 19, 958-961; Zwick, M. B., et al. (1998) Curr. Opin. Biotechnol. 9, 427-436) to identify peptides that bind to the cellular receptors ANTXR1 and ANTXR2. The I domains of ANTXR1 and ANTXR2 were purified and each was immobilized on plastic tubes, and then exposed to libraries of M13 phage. Each I domain was exposed to a phage library presenting random 12-residue, 7-residue, or constrained (cyclic) 7-residue peptides. In total, six screens were performed using the two I domains and the three libraries. After incubation of the phage with the I domain, the tube was washed to remove unbound and weakly bound phage; phage of interest were then eluted by adding PA. This protocol was repeated three times and the isolated phage were characterized by ELISA. The pool of phage selected from the 12-residue library after three rounds of panning bound the bait receptor, and did not bind to the unrelated protein bovine serum albumin (BSA) (data not shown).

We isolated a number of phage from the screens and tested each for the ability to bind the I domain that was used in the selection process (data not shown). Two 12-residue peptides that bind the I domain of ANTXR1 share the sequence QLDHS (Table 1).

TABLE 1

Sequence of peptides isolated from screening
phage display libraries against either ANTXR1
or ANTXR2

| | | | |
|---|---|---|---|
| ANTXR1-binding | SEQ | ANTXR2-binding | SEQ |
| peptides | ID | peptides | ID |

| | | | |
|---|---|---|---|
| AWPLSQLDHSYN* | 3 | SPHGSTDHSTTA* | 6 |
| YHLSSQQLDHSL* | 4 | STDHSLY‡ | 11 |
| ATWGHPRSSQGM* | 10 | STDSGWV‡ | 7 |
| CPSSTLFAC† | 9 | CTSTDATYC† | 8 |

Underlined sequences are common to at least two peptides. The sequence DHS, found in screens against both receptors, is in bold.
*Peptides isolated from 12-mer phage library.
†Peptides isolated from cyclic 7-mer peptide library.
‡Peptides isolated from 7-mer library.

Four peptides that bind ANTXR2 contain the sequence STD and two of these peptides, one from the 12-residue library and the other from the 7-residue library, shared an extended sequence, STDHS. Interestingly, the sequence DHS was shared by some of the peptides identified in screens against ANTXR1 and ANTXR2, indicating that these peptides might bind both receptors.

We synthesized four polyvalent inhibitors by attaching multiple copies of each DHS-containing peptide to liposome scaffolds, which are assemblies composed of a phospholipid bilayer and an aqueous core. Liposomes used for these experiments were made from a mixture of 1,2-distearoyl-sn-glycero-3-phosphocholine (DSPC) and a thiol-reactive pyridyldithiopropionate derivative of 1,2-dipalmitoyl-sn-glycero-3-phosphoethanolamine (PDP-DPPE) in a molar ratio of 9:1. The liposomes were functionalized with DHS-containing peptides with an additional C-terminal cysteine, which allowed attachment to the liposomes via a reaction with the thiol group. The remaining unreacted thiol-reactive groups on the liposomes were quenched with thioglycerol.

We tested the ability of these polyvalent liposome-based inhibitors to neutralize anthrax toxin in vitro by incubating RAW264.7 cells with a mixture of PA and LF in the presence of several concentrations of the inhibitor. As seen in Table 2, liposomes presenting AWPLSQLDHSYN (SEQ ID NO: 3), YHLSSQQLDHSL (SEQ ID NO: 4), SPHGSTDHSTTAY (SEQ ID NO: 5), and STDHSLY (SEQ ID NO: 11) (2.7% peptide) could inhibit the toxicity of a mixture of PA and LF in RAW264.7 cells.

TABLE 2
Inhibitory activities of polyvalent inhibitors

| | | |
|---|---|---|
| | | Activity against toxin- |
| | SEQ | induced cell death in |
| Peptide | ID | RAW264.7 cells (IC₅₀) |

| | | |
|---|---|---|
| AWPLSQLDHSYN | 3 | 40 nM |
| YHLSSQQLDHSL | 4 | 100 nM |
| SPHGSTDHSTTAY | 5 | 250 nM |
| STDHSLY | 11 | 900 nM |

Indicated peptides were attached to liposomes and their inhibitory activity was then measured. Values of IC₅₀ reported are on a per-peptide basis.

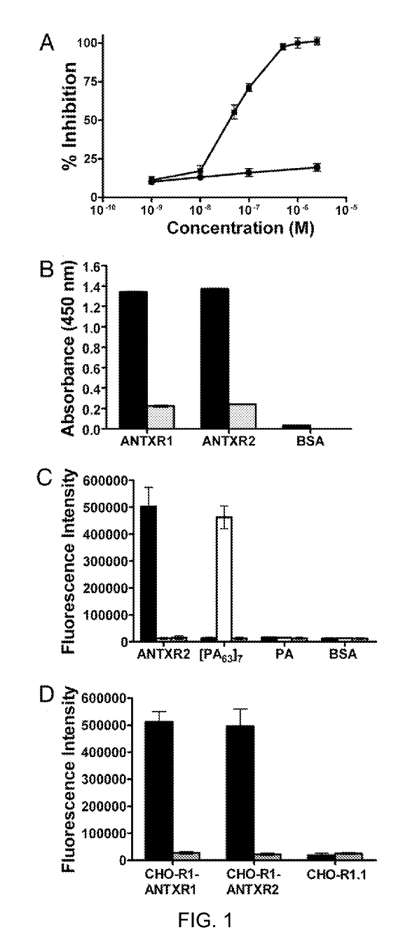

The most potent inhibitor, a liposome presenting multiple copies of AWPLSQLDHSYN, inhibited cytotoxicity with a half-maximal inhibitory concentration (IC₅₀) of 40 nM on a per-peptide basis (FIG. 1A); in contrast, the corresponding monovalent peptide did not inhibit cytotoxicity at concentrations as high as 2 mM. The use of polyvalency therefore enabled a more than 50,000-fold enhancement in the activity of this peptide. Liposomes presenting only thioglycerol showed no inhibitory activity (FIG. 1A).

Since the polyvalent inhibitor that displayed AWPLSQLDHSYN (SEQ ID NO: 3) was more potent than the others we tested, we decided to characterize this inhibitor further. This peptide was isolated from a screen for its ability to bind ANTXR1, but the protection of RAW264.7 cells by AWPLSQLDHSYN (SEQ ID NO: 3)-functionalized liposomes suggested that this peptide also binds ANTXR2 since ANTXR2 is expressed in RAW264.7 cells. (Rainey, G. J. A., et al. (2005) Proc. Natl. Acad. Sci. USA 102, 13278-13283) To test this possibility, we examined the binding of phage presenting the peptide AWPLSQLDHSYN (SEQ ID NO: 3) to ANTXR1 and ANTXR2 by ELISA. As seen in FIG. 1B, phage presenting this peptide bound to both receptors and did not bind to the unrelated protein BSA.

As a further test of the ability of the AWPLSQLDHSYN (SEQ ID NO: 3) peptide to bind the ANTXR2 I domain, we determined whether AWPLSQLDHSYN (SEQ ID NO: 3)-functionalized liposomes bound ANTXR2 in an ELISA. We attached the AWPLSQLDHSYN (SEQ ID NO: 3) peptide or a control [PA₆₃]₇-binding peptide, HTSTYWWLDGAP (SEQ ID NO: 12) (Mourez, M., Kane, et al. (2001) Nat. Biotechnol. 19, 958-961), to liposomes containing fluorescein (Peer, D. & Margalit, R. (2004) Int. J. Cancer 108, 780-789). Binding of fluorescein-containing liposomes to proteins adsorbed to plastic wells was detected using a fluorescence plate reader. Liposomes presenting AWPLSQLDHSYN (SEQ ID NO: 3) bound to ANTXR2, but did not bind to [PA₆₃]₇, PA, or BSA (FIG. 1C). In contrast, liposomes presenting HTSTYWWLDGAP (SEQ ID NO: 12) bound to [PA₆₃]₇ and not to the other three proteins (FIG. 1C). Liposomes presenting only thioglycerol did not bind to any of the proteins tested.

We next tested whether liposomes presenting AWPLSQLDHSYN (SEQ ID NO: 3) bind to full-length ANTXR1 and ANTXR2 in the physiologically-relevant context of the cell surface (FIG. 1D). This assay was carried out using the receptor-deficient CHO-R1.1 cell line and two cell lines derived from it: CHO-R1-ANTXR1 cells that express only ANTXR1 and CHO-R1-ANTXR2 cells that express only ANTXR2. Fluorescein-containing liposomes functionalized with AWPLSQLDHSYN bound to CHO-R1-ANTXR1 and CHO-R1-ANTXR2 cells, but not to CHO-R1.1 cells, whereas thioglycerol-functionalized fluorescein-containing liposomes did not bind to any of the three cell lines (FIG. 1D).

We also tested the ability of these receptor-directed polyvalent inhibitors to neutralize anthrax toxin in vivo, in Fisher 344 rats. Five of six rats that were injected intravenously with toxin (a mixture of 30 μg of PA and 8 μg of LF) and all six rats that were co-injected with toxin and unfunctionalized liposomes became moribund.

TABLE 3
Liposome inhibitors protect rats from LeTx challenge

| Treatment | Moribound rats/total |
|---|---|
| LeTx | 5/6 |
| LeTx + 400 nmol inhibitor | 0/6 |
| LeTx + thioglycerol-functionalized liposomes | 6/6 |

Rats were injected intravenously with only LeTx (30 μg of PA and 8 μg of LF), LeTx with thioglycerol-functionalized liposomes or LeTx with liposome inhibitors. The amount of inhibitor is listed in terms of amount of peptide. Results are combined from two independent experiments and are statistically significant by the log-rank test.

Co-injection of the AWPLSQLDHSYN (SEQ ID NO: 3)-functionalized liposomes with the toxin, however, prevented all six animals from becoming moribund. Six rats injected with the liposome-based inhibitors alone showed no adverse side effects.

To our knowledge, we have provided the first demonstration of the in vivo efficacy of a receptor-targeted anthrax anti-toxin. These anthrax toxin inhibitors might serve as valuable adjuncts to antibiotic therapy. While Bacillus anthracis can be eradicated from a host by treatment with antibiotics, such treatment is often insufficient to save the patient once symptoms have developed because of the continuing action of the secreted toxin. The administration of a receptor-directed polyvalent inhibitor could neutralize the toxin and help reduce the high mortality rates associated with inhalational anthrax.

Our approach to designing anthrax toxin receptor-directed inhibitors may be broadly applicable to receptors used by other pathogens and toxins (Mammen, M., et al. (1998) Angew. Chem. Int. Ed. 37, 2754-2794; Dhiman, N., et al. (2004) Rev. Med. Virol. 14, 217-229; Cywes, C., et al. (2000)J. Clin. Invest. 106, 995-1002; Li, W., et al. (2003) Nature 426, 450-454; Berger, E. A., et al. (1999) Annu. Rev. Immunol. 17, 657-700). Membrane proteins are currently the most highly represented class of drug targets for non-infectious diseases. Given the rapid emergence of antimicrobial drug resistance, targeting membrane receptors also represents a promising approach to design novel anti-infective agents. Phage display technology is inexpensive and allows for the rapid identification of peptides that can be used as the basis for polyvalent receptor-targeted inhibitors; synthesis of polyvalent inhibitors such as the ones described here and elsewhere is simple, inexpensive, and scalable, and provides significant enhancements in potency. Although broad-spectrum therapeutics have clear advantages, antimicrobials tailored to specific pathogens, especially those directed against host structures, may be an effective strategy to overcome the problem of resistance.

### Materials and Methods

Phage display selection. M13 phage libraries (New England Biolabs, Beverly, Mass.) displaying random 12-mer, 7-mer, and cyclic 7-mer peptides were used for panning. The I domains from ANTXR1 and ANTXR2 were purified as described previously (Lacy, D. B., et al. (2004) Proc. Natl. Acad. Sci. USA 101, 6367-637; Bradley, K. A., et al. (2003) J. Biol. Chem. 278, 49342-49347). Proteins were allowed to adsorb on Maxisorp tubes (Nunc, Roskilde, Denmark) from a 2 μg/mL solution, overnight at 4° C. The tubes were blocked with 2% BSA in PBS for 2 h and washed with PBS buffer. An M13 phage library (1.5×10¹¹ pfu in PBS) was added to the tubes and incubated for 1 h at room temperature. The tubes were then washed ten times with 0.1% Tween in PBS. The remaining bound phages were eluted by adding PA (20 μg/mL in PBS, 1 h incubation at room temperature). The amplified phage pool was panned again as described above except that the phages were added to the protein-coated tubes and incubated for 30 min in the second round and for five min in the third round, and the elution of bound phages was carried out by overnight incubation with PA (20 μg/mL in PBS) in rounds two and three.

Enzyme-linked Immunosorbent Assay. Purified receptor protein (I domain of ANTXR1 or ANTXR2) was coated in the wells of a 96-well plate overnight at 4° C. at 1 μg/mL in PBS (pH 7.5). The wells were blocked with 2% BSA in PBS for 2 h followed by a PBS wash. Phages (10⁸ pfu) were incubated in these protein-coated wells for 1 h. Phage binding to protein was quantified using an anti-phage antibody conjugated to horseradish peroxidase (Amersham Biosciences, Piscataway, N.J.) using 3,3′,5,5′-tetramethylbenzidine (Pierce Biotechnology, Rockford, Ill.) as a substrate. These experiments were carried out in duplicate and repeated twice.

Synthesis of liposome-based polyvalent inhibitor. Liposomes were made from a mixture of DSPC and the thiol-reactive lipid PDP-DPPE in a molar ratio of 9:1. Dynamic light scattering confirmed the presence of vesicles (radius 51±4 nm). Peptides identified by phage display were synthesized by Genemed Synthesis Inc (South San Francisco, Calif.). These peptides were acetylated at their N-terminus and amidated at the C-terminus, and had an extra cysteine residue at the C-terminus to facilitate their attachment to liposomes. Peptide pre-dissolved in DMSO was added to a solution of liposomes in phosphate buffer (pH 8) and the reaction was allowed to proceed overnight. The remaining unreacted thiol-reactive groups were quenched with thioglycerol. Excess unreacted peptide and thioglycerol were removed by dialysis. The peptide-functionalized liposomes were characterized by UV-Vis spectroscopy to determine the concentration of peptide (Mourez, M., Kane, et al. (2001) Nat. Biotechnol. 19, 958-961). The procedure used to prepare liposomes incorporating fluorescein was similar to that described above, except that the hydration step was carried out with a 10 mM solution of fluorescein in phosphate buffer (25 mM, pH 8). All liposomes used in this study were functionalized with 2.7% peptide. Inhibitor concentrations are on a per peptide basis.

Characterization of the Binding of Liposomes to Immobilized Proteins. Purified protein was coated in the wells of a 96-well plate overnight at 4° C. from a 10 μg/mL solution in PBS (pH 7.4). The wells were blocked with 100 μl of 2% BSA in PBS for 2 h. The fluorescein-containing liposomes were then incubated in the wells for 1 h at room temperature. The wells were then washed 10 times with PBS to remove any unbound liposomes. Finally, the intensity of fluorescence in the wells was quantified using a plate reader (Perkin Elmer). Excitation was at 495 nm and emission was read at 535 nm with a measurement time of 1 s. Results reported here are the average of 3 independent experiments, each carried out in triplicate.

Characterization of the Binding of Liposomes to Cells. The binding experiment was carried out using CHO-R1-ANTXR1, CHO-R1-ANTXR2, and receptor-deficient CHO-R1.1 cells. Cells were cultured in F12 medium supplemented with 10% fetal bovine serum (FBS) (Invitrogen). The cells were seeded in a 96-well plate at a density of 10,000 cells per well and incubated overnight. After washing with PBS, the wells were blocked by adding 100 μl of a solution of 5% Bovine Serum Albumin (BSA) in PBS for 2 h. Unbound BSA was removed by washing the wells with PBS. Next, the cells were incubated with 100 μl of 50 mM 2-deoxy-glucose (Sigma)/10 mM sodium azide (Sigma)/200 nM bafilomycin A1 (Alexis Biochemicals) (dGAB) in F12 medium supplemented with 10% FBS for 45 min at 37° C. (18). The fluorescein-containing liposomes were then added to the wells and incubated for 1 h at room temperature. After 10 washes with PBS, the values of the fluorescence intensity in the wells were measured using a plate reader (Perkin Elmer). Excitation was at 495 nm and emission was read at 535 nm with a measurement time of 1 s. Results reported here are the average of 3 independent experiments, each carried out in triplicate.

Cytotoxicity Assay. RAW264.7 cells were seeded in 96-well plates and incubated overnight. The cells were treated with 10⁻⁹ M PA and 3×10⁻¹⁰ M LF in the absence or presence of inhibitors. After an incubation period of 4 h, cell viability was assessed using the MTS assay according to the manufacturer's instructions (Promega).

Rat Intoxication. A mixture of purified PA (30 μg) and LF (8 μg) mixed with either PBS, peptide-functionalized liposomes (2.7% peptide; 400 nmol on a per-peptide basis), or thioglycerol-functionalized liposomes, was used for the intoxication assay. Fisher 344 rats (Charles River Laboratories) were injected intravenously in the tail vein. Three rats were used per group, and the appearance of symptoms of intoxication was monitored over 4 h. The rats were euthanized to avoid unnecessary distress when the symptoms became pronounced.

While this invention has been particularly shown and described with references to preferred embodiments thereof, it will be understood by those skilled in the art that various changes in form and details may be made therein without departing from the scope of the invention encompassed by the appended claims.

1. A polyvalent inhibitor comprising a biocompatible scaffold having attached thereto a plurality of ligands capable of binding a host cell receptor or portion thereof of a pathogen or toxin of a pathogen.

2. The polyvalent inhibitor of claim 1 wherein the pathogen is Corynebacterium diphtheriae, Bordetella pertussis, Vibrio cholerae, Bacillus anthracis, Clostridium botulinum, Clostridium tetani, and enterohemorrhagic Escherichia coli.

3. The inhibitor of claim 1 wherein the scaffold is a liposome or polymer.

4. The inhibitor of claim 1 wherein the scaffold is a liposome comprising at least one lipid having a head group covalently bound to each ligand.

5. The inhibitor of claim 4 wherein each ligand is a peptide between about 3 to about 30 amino acids in length.

6. The inhibitor of claim 5 wherein at least one of the peptides is about 5 to about 15 amino acids in length.

7. The inhibitor of claim 6 wherein at least one peptide is about 7 to about 13 amino acids in length.

8. The inhibitor of claim 1 wherein the pathogen is Bacillus anthracis and the toxin is anthrax.

9. The inhibitor of claim 8 wherein the receptor of the pathogen or toxin is ANTXR1 and ANTXR2.

10. The inhibitor of claim 8 wherein the receptor of the pathogen or toxin is the I domain of ANTXR1 or ANTXR2.

11. The inhibitor of claim 10 wherein the at least one ligand is capable of binding all or a portion of the I domain of ANTXR1, or ANTXR2 or both.

12. The inhibitor of claim 5 wherein at least one peptide comprises the amino acid sequence STD.

13. The inhibitor of claim 5 wherein at least one peptide comprises the amino acid sequence STDHS.

14. The inhibitor of claim 5 wherein at least one peptide comprises the amino acid sequence DHS.

15. The inhibitor of claim 5 wherein at least one peptide comprises an amino acid sequence selected from the group consisting of:

| | |
|---|---|
| AWPLSQLDHSYN, | (SEQ ID NO. 3) |
| YHLSSQQLDHSL, | (SEQ ID NO. 4) |
| SPHGSTDHSTTAY, | (SEQ ID NO. 5) |
| SPHGSTDHSTTA, | (SEQ ID NO. 6) |
| STDSGWV, | (SEQ ID NO. 7) |
| CTSTDATYC, | (SEQ ID NO. 8) |
| CPSSTLFAC, | (SEQ ID NO. 9) |
| ATWGHPRSSQGM, | (SEQ ID NO. 10) |
| and | |
| STDHSLY. | (SEQ ID NO. 11) |

16. The inhibitor of claim 15 wherein at least one peptide of the plurality of peptides comprises the amino acid sequence AWPLSQLDHSYN (SEQ ID NO. 3).

17. The inhibitor of claim 5 wherein the peptide is present in multiple copies.

18. A method of treating a condition in a patient caused by a pathogen or a toxin of a pathogen comprising administering to the patient an effective amount of the polyvalent inhibitor of claim 1.

19. A method of treating a patient susceptible to a condition caused by a pathogen or a toxin of a pathogen comprising administering to the patient an effective amount of the polyvalent inhibitor of claim 5.

20. The method of claim 19 wherein the pathogen is Bacillus anthracis and the toxin is anthrax.

21. The method of claim 19 wherein the receptor of the pathogen or toxin is ANTXR1 and ANTXR2.

22. The method of claim 19 wherein the receptor of the pathogen or toxin is the I domain of ANTXR1 or ANTXR2.

23. The method of claim 19 wherein the at least one peptide is capable of binding all or a portion of the I domain of ANTXR1, or ANTXR2 or both.

24. The method of claim 19 wherein at least one peptide comprises the amino acid sequence STD.

25. The method of claim 19 wherein at least one peptide comprises the amino acid sequence STDHS.

26. The method of claim 19 wherein at least one peptide comprises the amino acid sequence DHS.

27. The method of claim 19 wherein at least one peptide comprises an amino acid sequence selected from the group consisting of:

| | |
|---|---|
| AWPLSQLDHSYN, | (SEQ ID NO. 3) |
| YHLSSQQLDHSL, | (SEQ ID NO. 4) |
| SPHGSTDHSTTAY, | (SEQ ID NO. 5) |
| SPHGSTDHSTTA, | (SEQ ID NO. 6) |
| STDSGWV, | (SEQ ID NO. 7) |
| CTSTDATYC, | (SEQ ID NO. 8) |
| CPSSTLFAC, | (SEQ ID NO. 9) |
| ATWGHPRSSQGM, | (SEQ ID NO. 10) |
| and | |
| STDHSL. | (SEQ ID NO. 11) |

28. A method of treating an infection in a patient by a multi-drug resistant pathogen comprising administering to the patient an effective amount of the polyvalent inhibitor of claim 1.

29. A method for designing a polyvalent pathogen inhibitor comprising the steps of:
(a) providing a purified host cell receptor or portion thereof of a pathogen or toxin of the pathogen;
(b) contacting the receptor of step (a) with a phage library displaying random peptides and isolating the phages that bind to the receptor of step (a);
(c) identifying the peptide associated with the phages isolated in step (b); and
(d) attaching multiple copies of the peptide identified in step (c) to a scaffold;
wherein the polyvalent inhibitor is capable of inhibiting the binding of a pathogen or a toxin of a pathogen to the host cell's receptor for the pathogen or toxin of the pathogen.

30. The method of claim 29 wherein the pathogen is Corynebacterium diphtheriae (diphtheria), Bordetella pertussis (whooping cough), Vibrio cholerae (cholera), Bacillus anthracis (anthrax), Clostridium botulinum (botulism), Clostridium tetani (tetanus), and enterohemorrhagic Escherichia coli (bloody diarrhea and hemolytic uremic syndrome).

31. The method of claim 29 wherein the pathogen is Bacillus anthracis (anthrax).

32. The method of claim 29 wherein the scaffold is a liposome or polymer.
